(19) United States
(12) Patent Application Publication
(10) Pub. No.: US 2026/0201004 A1
(43) Pub. Date: Jul. 16, 2026

(54) ENGINEERED PLATELET-DERIVED GROWTH FACTOR VARIANTS

(71) Applicant: MULTUS BIOTECHNOLOGY LIMITED, London (GB)

(72) Inventors: Kevin PAN, London (GB); Seong Hyun Brandon MA, London (GB); Morgane VALLES, London (GB); George Mackford TAYLOR, London (GB)

(21) Appl. No.: 19/138,313

(22) PCT Filed: Dec. 15, 2023
(86) PCT No.: PCT/EP2023/086166

§ 371 Date: Jun. 12, 2025

(30) Foreign Application Priority Data
Dec. 15, 2022 (GB) 2218987.2

(51) Int. Cl. C07K 14/49; C12N 5/00

(52) U.S. Cl. CPC C07K 14/49; C12N 5/0018; C12N 2501/135

(57) ABSTRACT

The invention relates to improved platelet-derived growth factor (PDGF) polypeptide variants, in particular polypeptides comprising a sequence of any one of SEQ ID NOs: 2-4 or a sequence with at least 80% similarity thereto, wherein the PDGF polypeptide exhibits increased stability, bioactivity and/or solubility compared to the wild-type PDGFB polypeptide of SEQ ID NO: 1. The invention further provides nucleic acid sequences encoding a PDGF polypeptide of the invention, and a method of growing an animal cell comprising cultivating the animal cell in a culture medium comprising a PDGF polypeptide of the invention is also provided.

### PRIORITY STATEMENT

This application is a national stage application under 35 U.S.C. § 371 of PCT International Patent Application No. PCT/EP2023/086166, which has an international filing date of 15 Dec. 2023 and claims priority under 35 U.S.C. § 119 to GB Patent Application No. 2218987.2 filed on 15 Dec. 2022. The content of each application recited above is incorporated herein by reference in its entirety.

### REFERENCE TO AN ELECTRONIC SEQUENCE LISTING

This application contains references to amino acid sequences and/or nucleic acid sequences which have been submitted concurrently herewith as the sequence listing.xml file entitled “19306-000004-US-NP_8_May_2025_ST26.xml” file size 8,482 Bytes (B), created on 8 May 2025. The aforementioned sequence listing is hereby incorporated by reference in its entirety.

### FIELD OF THE INVENTION

The invention relates to improved growth factor polypeptide variants, in particular variants of platelet-derived growth factors (PDGF) and uses thereof, including use in cell culture media.

### BACKGROUND OF THE INVENTION

Growth factors are naturally occurring cell signalling molecules that play a number of essential roles including regulating cell proliferation and development and cellular differentiation.

One of the most common mechanisms for controlling cell signals in organisms is decay of the signalling molecule. Hence, most wild-type growth factors have high turnover rates and are not “built” to last, making them less suitable for the industrial setting where a constant level of stimulus for cell proliferation should be maintained.

There are a number of disadvantages related to this rapid degradation of wild-type growth factors, including the wasteful and inefficient use of components and introduction of variability in the context of cell culture.

Growth medium used in cell culture usually includes combinations of growth factors. To overcome the exponential depletion of these naturally occurring cell growth signalling stimuli, high levels of growth factors are used to keep the stimulus above a minimal threshold and the media must be replaced frequently (for example, every two days) even where there are still enough nutrients.

In the growing field of cultivated meat, cell culture is a fundamental aspect of the process. One of the limiting steps in the production of cultivated meat is the high cost of cell growth media. Climate and other environmental concerns are continuing to drive the demand for cultivated meat, and therefore also the need for improved growth media to replace animal serum-based media.

To address these issues, a series of engineered polypeptides are provided with improved properties, such as increased stability (including thermostability). These polypeptides can be used as a replacement for naturally occurring wild-type growth factors in growth media. The improved properties also make the polypeptides useful in a number of other areas.

Platelet-derived growth factors (PDGF) are a family of cell signalling proteins, and members of this family are involved in a wide variety of cellular processes. PDGF polypeptides are commonly used in growth media and there is an ongoing need for PDGF polypeptides with improved properties, such as increased stability, bioactivity and/or solubility. PDGFB is one member of this family and binds to specific PDGF receptors. PDGFB can exist as a homodimer (also known as PDGFbb) or a heterodimer with PDGFA (known as PDGFab). PDGFB can stimulate proliferation and differentiation of various cell types, and aid in the process of angiogenesis.

The present invention meets this need by providing novel polypeptides with improved properties.

### SUMMARY OF THE INVENTION

Provided herein is a platelet-derived growth factor (PDGF) polypeptide comprising at least one modification selected from the group consisting of an amino acid substitution, an amino acid deletion, an amino acid insertion, and combinations thereof relative to the sequence set forth in SEQ ID NO: 1, wherein the modified PDGF polypeptide exhibits increased stability (including thermostability), bioactivity and/or solubility compared to the PDGFB polypeptide of SEQ ID NO: 1.

Also provided herein is a PDGF polypeptide comprising a sequence of any one of SEQ ID NOs: 2-4 or a sequence with at least 80% similarity thereto, wherein the PDGF polypeptide exhibits increased stability (including thermostability), bioactivity and/or solubility compared to the wild-type PDGFB polypeptide of SEQ ID NO: 1.

Also provided herein is a nucleic acid sequence encoding a PDGF polypeptide of the invention.

Further provided is a cell comprising a polypeptide of the invention or a nucleic acid sequence of the invention.

A method of growing an animal cell comprising cultivating the animal cell in a culture medium comprising a PDGF polypeptide of the invention is also provided.

### BRIEF DESCRIPTION OF THE FIGURES

FIG. 1 shows alignment of the amino acid sequence of polypeptides of the invention (SEQ ID NOs: 2-4).

FIG. 2 shows alignment of the amino acid sequence of polypeptides of the invention (SEQ ID NOs: 2-4) and the wild-type PDGFB sequence (SED ID NO: 1).

FIGS. 3A-3C illustrate schematic views of the plasmid insertions encoding the polypeptides of SEQ ID NOS: 2-4.

FIGS. 4A and 4B illustrate the results of sodium dodecyl sulfate-polyacrylamide gel electrophoresis (SDS-PAGE) and western blot analyses, which show expression of the polypeptides of the invention.

FIGS. 5A-5C show a comparison of receptor kinase activity (bioactivity) of PDGF polypeptides of the invention compared to wild-type PDGFB. The grey lines represent polypeptides of the invention and the black line represents wild-type PDGFB.

### DETAILED DESCRIPTION OF THE INVENTION

The present invention provides novel PDGF polypeptide sequences, including PDGFB polypeptide sequences, with improved properties over known PDGF polypeptides, such as increased stability, including increased thermostability (an increased melting temperature (Tm)). The present invention also provides nucleic acids, encoding the PDGF polypeptides of the present invention.

Unless defined otherwise, all technical and scientific terms used herein generally have the same meaning as commonly understood by a person skilled in the art to which this invention belongs. Generally, the nomenclature used herein and the laboratory procedures in cell culture, molecular genetics, organic chemistry, nucleic acid chemistry and hybridisation are those well-known and commonly employed in the art. Standard techniques are used for nucleic acid and peptide synthesis. The techniques and procedures are generally performed according to conventional methods in the art. The nomenclature used herein, and the laboratory procedures of synthetic biology described below, are those well-known and commonly employed in the art.

### Polypeptides of the Invention

In some embodiments, the invention provides a polypeptide comprising at least one modification selected from the group consisting of an amino acid substitution, an amino acid deletion, an amino acid insertion, and combinations thereof relative to the sequence set forth in SEQ ID NO: 1, wherein the modified polypeptide exhibits increased stability compared to the PDGFB polypeptide of SEQ ID NO: 1.

In some embodiments, the invention provides a polypeptide comprising at least one modification selected from the group consisting of an amino acid substitution, an amino acid deletion, an amino acid insertion, and combinations thereof relative to the sequence set forth in SEQ ID NO: 1, wherein the modified polypeptide is bioactive, and exhibits changes to stability and/or solubility, compared to the PDGFB polypeptide of SEQ ID NO: 1.

In some embodiments, the invention provides a polypeptide comprising at least one modification selected from the group consisting of an amino acid substitution, an amino acid deletion, an amino acid insertion, and combinations thereof relative to the sequence set forth in SEQ ID NO: 1, wherein the modified polypeptide is bioactive and exhibits stability and/or solubility at a level comparable to that exhibited by the PDGFB polypeptide of SEQ ID NO: 1.

In some embodiments, the polypeptide of the invention is a PDGF polypeptide.

In some embodiments, the polypeptide of the invention is a PDGFB polypeptide.

In some embodiments, the polypeptide of the invention is a dimer, including a homodimer.

In some embodiments, the polypeptide of the invention binds at least one platelet-derived growth factor receptor (PDGFR).

In some embodiments, the polypeptide of the invention binds at least PDGFRB. This receptor is also known as PDGFR beta.

In some embodiments, the polypeptide of the present invention is a recombinant polypeptide, a natural polypeptide or a synthetic polypeptide, preferably a recombinant polypeptide.

The substitution may be a conservative amino acid substitution, meaning a substitution of one amino acid residue for another sharing chemical and physical properties of the amino acid side chain (e.g., charge, size, hydrophobicity/hydrophilicity). Conservative substitutions are intended to include substitution within the following groups of amino acid residues: gly, ala; val, ile, leu; asp, glu; asn, gin; ser, thr; lys, arg; and phe, tyr.

The terms ‘wild-type PDGFB’, ‘WT PDGFB’, ‘naturally occurring PDGFB’ or ‘natural PDGFB’ refer to PDGFB that occurs in nature, without modification.

Human wild-type PDGFB protein comprises the sequence of SEQ ID NO: 1, and is shown below:

SLGSLTIAEPAMIAECKTRTEVFEISRRLIDRTNANFLVWPPCVE
VQRCSGCCNNRNVQCRPTQVQLRPVQVRKIEIVRKKPIFKKATVT
LEDHLACKCETVAAARPVT

PDGFB has a number of functions, such as stimulating proliferation and differentiation of various cell types, and can aid in the angiogenesis process.

PDGFB binds to platelet-derived growth factor receptors (PDGFRs), stimulating the receptors. This bioactivity is required for effective function of PDGFB proteins, both for naturally occurring and engineered varieties of PDGFB.

In some embodiments, the polypeptide of the invention exhibits increased bioactivity compared to the PDGFB polypeptide of SEQ ID NO: 1. In some embodiments, the polypeptide of the invention exhibits increased stability (for example, increased thermostability) compared to the PDGFB polypeptide of SEQ ID NO: 1. In some embodiments, the polypeptide of the invention exhibits increased solubility, for example in Escherichia coli (E. coli) or yeast, compared to the PDGFB polypeptide of SEQ ID NO: 1. In some embodiments, the polypeptide of the invention exhibits both increased stability (for example, increased thermostability) and increased bioactivity compared to the PDGFB polypeptide of SEQ ID NO: 1. In some embodiments, the polypeptide of the invention exhibits increased stability (for example, increased thermostability), increased bioactivity and increased solubility compared to the PDGFB polypeptide of SEQ ID NO: 1.

A preferred approach for measuring stability, including thermostability, is by assessing bioactivity over time, such as by using the ONE-Glo Luciferase assay (Promega). ONE-Glo luciferase assay detects and quantifies expression of a luciferase reporter gene, in response to a signal transduction triggered by PDGF binding to the PDGFR. This assay is described in detail in Example 6 and also in Example 3.

Melting temperature of a polypeptide can also be used to quantify thermostability. Wild-type PDGFB polypeptide has a melting temperature (Tm) of about 50° C. Thermostability can be measured by Differential Scanning Fluorometry, where changes in fluorescence of a dye that binds preferentially to unfolded protein over folded protein, for example Spyro Orange, are measured by PCR, for example, real time PCR, and a melting temperature (° C.) is determined.

In some embodiments, the polypeptide of the invention exhibits a melting temperature comparable to the melting temperature of the PDGFB polypeptide of SEQ ID NO: 1. In this context, comparable may mean within 20° C., within 10° C., or within 5° C.

In some embodiments, the polypeptide of the invention exhibits a melting temperature higher than the melting temperature of the PDGFB polypeptide of SEQ ID NO: 1. The increase in melting temperature can be an increase of approximately 1° C., 2° C., 3° C., 4° C., 5° C., 10° C., 15° C., 20° C., 25° C. or 30° C.

The term “bioactive” refers to the ability of a polypeptide to bind a relevant receptor. Bioactivity as used herein can refer to the ability of a polypeptide of the invention to bind a platelet-derived growth factor receptor (PDGFR), such as PDGFRB. Bioactivity can be determined by a ONE-Glo Luciferase assay (Promega). This assay is described in detail in Example 3. EC50 (half maximal effective concentration) quantifies bioactivity.

In some embodiments, a polypeptide of the invention has a lower EC50 than the PDGFB polypeptide of SEQ ID NO: 1 when the EC50 of each polypeptide is measured under the same conditions.

It will be understood that in order to compare the EC50 of a polypeptide of the invention to the EC50 of the PDGFB polypeptide of SEQ ID NO: 1, the EC50 of both are measured in the same experiment and under the same conditions. Therefore, in an embodiment, improved bioactivity of a polypeptide of the invention means a lower EC50 than the PDGFB polypeptide of SEQ ID NO: 1, when measured under the same conditions.

In some embodiments, the polypeptide of the invention has an EC50 at least 2-fold lower than the EC50 of the wild-type PDGFB polypeptide of SEQ ID NO: 1. In some embodiments, the polypeptide of the invention has an EC50 at least 5-fold lower than the EC50 of the wild-type PDGFB polypeptide of SEQ ID NO: 1. In some embodiments, the polypeptide of the invention has an EC50 at least 10-fold lower than the EC50 of the wild-type PDGFB polypeptide of SEQ ID NO: 1.

In some embodiments, the polypeptide of the invention has an EC50 at least 0.05 ng/ml below the EC50 of the wild-type PDGFB polypeptide of SEQ ID NO: 1. In some embodiments, the polypeptide of the invention has an EC50 at least 0.075 ng/ml below that of the wild-type PDGFB polypeptide of SEQ ID NO: 1. In some embodiments, the polypeptide of the invention has an EC50 at least 0.1 ng/ml below that of the wild-type PDGFB polypeptide of SEQ ID NO: 1. In some embodiments, the polypeptide of the invention has an EC50 at least 0.125 ng/ml below that of the wild-type PDGFB polypeptide of SEQ ID NO: 1. In some embodiments, the polypeptide of the invention has an EC50 at least 0.15 ng/ml below that of the wild-type PDGFB polypeptide of SEQ ID NO: 1.

In some embodiments, the polypeptide of the invention is capable of binding a platelet-derived growth factor receptor (PDGFR). In some embodiments, the polypeptide of the invention is capable of binding to PDGFRA and/or PDGFRB. In some embodiments, the polypeptide of the invention is capable of binding to PDGFRA and PDGFRB.

“Solubility” as used herein refers to ability of the polypeptide to fold into a functional protein. Solubility can be an indication of expression level of functional protein by a particular expression platform. Solubility can be determined by using SDS-PAGE and western blot. This assay is described in detail in Example 4. Soluble fraction (%) quantifies solubility.

The wild-type PDGFB polypeptide of SEQ ID NO: 1 is not soluble. Therefore, it is an object of the present invention to provide soluble variants. In an embodiment, the polypeptide of the invention is soluble.

An insoluble protein requires a denaturing purification, which unfolds and then re-folds the protein during the purification. The re-folding step is inefficient and sometimes does not work. In contrast, a soluble protein (such as the polypeptides of the present invention) can be purified by a native purification, which does not require unfolding the protein. This is one advantage of the present invention.

In some embodiments, the polypeptide of the invention has a solubility of at least 30%. In some embodiments, the polypeptide of the invention has a solubility of at least 40%. In some embodiments, the polypeptide of the invention has a solubility of at least 50%. In some embodiments, the polypeptide of the invention has a solubility of at least 60%. In some embodiments, the polypeptide of the invention has a solubility of at least 70%. In some embodiments, the polypeptide of the invention has a solubility of at least 80%. In some embodiments, the polypeptide of the invention has a solubility of at least 90%. In some embodiments, the polypeptide of the invention has a solubility of 100%.

In some embodiments, the polypeptide of the invention is more stable than wild-type PDGFB (e.g. a polypeptide of SEQ ID NO: 1) and has higher or substantially the same bioactivity levels. In some embodiments, the polypeptide of the invention is more stable than wild-type PDGFB (e.g. a polypeptide of SEQ ID NO: 1) and exhibits substantially the same solubility.

In some embodiments, the polypeptide of the invention is soluble and bioactive. In some embodiments, the polypeptide of the invention is soluble, bioactive and thermostable between 39° C. and 49° C. In some embodiments, the polypeptide is soluble, bioactive and thermostable at 40° C.

In some embodiments, the polypeptide of the invention is soluble and exhibits substantially the same stability as the PDGFB polypeptide of SEQ ID NO: 1, or higher stability. In some embodiments, the polypeptide of the invention is bioactive and exhibits increased stability compared to the PDGFB polypeptide of SEQ ID NO: 1. In some embodiments, the polypeptide of the invention is soluble and bioactive and exhibits increased stability compared to the PDGFB polypeptide of SEQ ID NO: 1.

In some embodiments, the polypeptide of the invention comprises a sequence of any one of SEQ ID NOs: 2-4.

In some embodiments, the polypeptide of the invention consists of the sequence of any one of SEQ ID NOs: 2-4.

In some embodiments, the polypeptide of the invention comprises a sequence with at least 80% similarity to any one of SEQ ID NOs: 2-4. In some embodiments, the polypeptide of the invention comprises a sequence with at least 85% similarity to any one of SEQ ID NOs: 2-4. In some embodiments, the polypeptide of the invention comprises a sequence with at least 90% similarity to any one of SEQ ID NOs: 2-4. In some embodiments, the polypeptide of the invention comprises a sequence with at least 92% similarity to any one of SEQ ID NOs: 2-4. In some embodiments, the polypeptide of the invention comprises a sequence with at least 95% similarity to any one of SEQ ID NOs: 2-4. The polypeptide of the embodiment can be a PDGFB polypeptide. The polypeptide of this embodiment can bind at least one platelet-derived growth factor receptor (PDGFR).

In some embodiments, the polypeptide of the invention comprises a sequence with at least 96% similarity to any one of SEQ ID NOs: 2-4. In some embodiments, the polypeptide of the invention comprises a sequence with at least 97% similarity to any one of SEQ ID NOs: 2-4. In some embodiments, the polypeptide of the invention comprises a sequence with at least 98% similarity to any one of SEQ ID NOs: 2-4. In some embodiments, the polypeptide of the invention comprises a sequence with at least 99% similarity to any one of SEQ ID NOs: 2-4. The polypeptide of the embodiment can be a PDGFB polypeptide. The polypeptide of this embodiment can bind at least one platelet-derived growth factor receptor (PDGFR).

In a preferred embodiment, the polypeptide of the invention comprises a sequence with at least 90% similarity to SEQ ID NO: 2 or SEQ ID NO: 4.

In a preferred embodiment, the polypeptide of the invention comprises a sequence with at least 92% similarity to SEQ ID NO: 3.

In a preferred embodiment, the polypeptide of the invention comprises a sequence with at least 95% similarity to any one of SEQ ID NOs: 2-4.

Percent similarity (or ‘percentage similarity’) between two sequences can be calculated by multiplying the number of matches in the pair by 100 and dividing by the length of the aligned region, including gaps. Identity scoring only counts perfect matches and does not consider the degree of similarity of amino acids to one another. Gaps at the end of sequences are not included, and internal gaps are included in the length. Alignments can be generated using programs known in the art. For purposes herein, alignment of nucleotide sequences can be performed with the blastn program set at default parameters, and alignment of amino acid sequences can be performed with the blastp program set at default parameters (see National Center for Biotechnology Information (NCBI): ncbi.nlm.nih.gov).

In some embodiments, the polypeptide of the invention comprises the sequence of SEQ ID NO: 2 or SEQ ID NO: 4. In some embodiments, the polypeptide of the invention comprises a sequence with at least 80% similarity to the sequence of SEQ ID NO: 2 or SEQ ID NO: 4. In some embodiments, the polypeptide of the invention comprises a sequence with at least 85% similarity to the sequence of SEQ ID NO: 2 or SEQ ID NO: 4. In some embodiments, the polypeptide of the invention comprises a sequence with at least 90% similarity to the sequence of SEQ ID NO: 2 or SEQ ID NO: 4. In some embodiments, the polypeptide of the invention comprises a sequence with at least 92% similarity to the sequence of SEQ ID NO: 2 or SEQ ID NO: 4. In some embodiments, the polypeptide of the invention comprises a sequence with at least 95% similarity to the sequence of SEQ ID NO: 2 or SEQ ID NO: 4. In some embodiments, the polypeptide of the invention consists of the sequence of SEQ ID NO: 2 or SEQ ID NO: 4.

In some embodiments, the polypeptide of the invention comprises a sequence with at least 96% similarity to the sequence of SEQ ID NO: 2 or SEQ ID NO: 4. In some embodiments, the polypeptide of the invention comprises a sequence with at least 97% similarity to the sequence of SEQ ID NO: 2 or SEQ ID NO: 4. In some embodiments, the polypeptide of the invention comprises a sequence with at least 98% similarity to the sequence of SEQ ID NO: 2 or SEQ ID NO: 4. In some embodiments, the polypeptide of the invention comprises a sequence with at least 99% similarity to the sequence of SEQ ID NO: 2 or SEQ ID NO: 4.

In some embodiments, the polypeptide of the invention comprises the sequence of SEQ ID NO: 2. In some embodiments, the polypeptide of the invention comprises a sequence with at least 80% similarity to the sequence of SEQ ID NO: 2. In some embodiments, the polypeptide of the invention comprises a sequence with at least 85% similarity to the sequence of SEQ ID NO: 2. In some embodiments, the polypeptide of the invention comprises a sequence with at least 90% similarity to the sequence of SEQ ID NO: 2. In some embodiments, the polypeptide of the invention comprises a sequence with at least 92% similarity to the sequence of SEQ ID NO: 2. In some embodiments, the polypeptide of the invention comprises a sequence with at least 95% similarity to the sequence of SEQ ID NO: 2. In this embodiment, the polypeptide of the invention can be more stable than wild-type PDGFB (e.g. a polypeptide of SEQ ID NO: 1). In some embodiments, the polypeptide of the invention consists of the sequence of SEQ ID NO: 2.

In some embodiments, the polypeptide of the invention comprises a sequence with at least 96% similarity to the sequence of SEQ ID NO: 2. In some embodiments, the polypeptide of the invention comprises a sequence with at least 97% similarity to the sequence of SEQ ID NO: 2. In some embodiments, the polypeptide of the invention comprises a sequence with at least 98% similarity to the sequence of SEQ ID NO: 2. In some embodiments, the polypeptide of the invention comprises a sequence with at least 99% similarity to the sequence of SEQ ID NO: 2. In this embodiment, the polypeptide of the invention can be more stable than wild-type PDGFB (e.g. a polypeptide of SEQ ID NO: 1).

In a preferred embodiment, the polypeptide of the invention comprises a sequence with at least 92% similarity to the sequence of SEQ ID NO: 2.

In a preferred embodiment, the polypeptide of the invention comprises a sequence with at least 95% similarity to the sequence of SEQ ID NO: 2.

In some embodiments, the polypeptide of the invention comprises the sequence of SEQ ID NO: 3. In some embodiments, the polypeptide of the invention comprises a sequence with at least 80% similarity to the sequence of SEQ ID NO: 3. In some embodiments, the polypeptide of the invention comprises a sequence with at least 85% similarity to the sequence of SEQ ID NO: 3. In some embodiments, the polypeptide of the invention comprises a sequence with at least 90% similarity to the sequence of SEQ ID NO: 3. In some embodiments, the polypeptide of the invention comprises a sequence with at least 92% similarity to the sequence of SEQ ID NO: 3. In some embodiments, the polypeptide of the invention comprises a sequence with at least 95% similarity to the sequence of SEQ ID NO: 3. In this embodiment, the polypeptide of the invention can be more stable than wild-type PDGFB (e.g. a polypeptide of SEQ ID NO: 1). In some embodiments, the polypeptide of the invention consists of the sequence of SEQ ID NO: 3.

In some embodiments, the polypeptide of the invention comprises a sequence with at least 96% similarity to the sequence of SEQ ID NO: 3. In some embodiments, the polypeptide of the invention comprises a sequence with at least 97% similarity to the sequence of SEQ ID NO: 3. In some embodiments, the polypeptide of the invention comprises a sequence with at least 98% similarity to the sequence of SEQ ID NO: 3. In some embodiments, the polypeptide of the invention comprises a sequence with at least 99% similarity to the sequence of SEQ ID NO: 3. In this embodiment, the polypeptide of the invention can be more stable than wild-type PDGFB (e.g. a polypeptide of SEQ ID NO: 1).

In a preferred embodiment, the polypeptide of the invention comprises a sequence with at least 92% similarity to the sequence of SEQ ID NO: 3.

In a preferred embodiment, the polypeptide of the invention comprises a sequence with at least 95% similarity to the sequence of SEQ ID NO: 3.

In some embodiments, the polypeptide of the invention comprises the sequence of SEQ ID NO: 4. In some embodiments, the polypeptide of the invention comprises a sequence with at least 80% similarity to the sequence of SEQ ID NO: 4. In some embodiments, the polypeptide of the invention comprises a sequence with at least 85% similarity to the sequence of SEQ ID NO: 4. In some embodiments, the polypeptide of the invention comprises a sequence with at least 90% similarity to the sequence of SEQ ID NO: 4. In some embodiments, the polypeptide of the invention comprises a sequence with at least 92% similarity to the sequence of SEQ ID NO: 4. In some embodiments, the polypeptide of the invention comprises a sequence with at least 95% similarity to the sequence of SEQ ID NO: 4. In this embodiment, the polypeptide of the invention can be more stable than wild-type PDGFB (e.g. a polypeptide of SEQ ID NO: 1). In some embodiments, the polypeptide of the invention consists of the sequence of SEQ ID NO: 4.

In some embodiments, the polypeptide of the invention comprises a sequence with at least 96% similarity to the sequence of SEQ ID NO: 4. In some embodiments, the polypeptide of the invention comprises a sequence with at least 97% similarity to the sequence of SEQ ID NO: 4. In some embodiments, the polypeptide of the invention comprises a sequence with at least 98% similarity to the sequence of SEQ ID NO: 4. In some embodiments, the polypeptide of the invention comprises a sequence with at least 99% similarity to the sequence of SEQ ID NO: 4. In this embodiment, the polypeptide of the invention can be more stable than wild-type PDGFB (e.g. a polypeptide of SEQ ID NO: 1).

In a preferred embodiment, the polypeptide of the invention comprises a sequence with at least 92% similarity to the sequence of SEQ ID NO: 4.

In a preferred embodiment, the polypeptide of the invention comprises a sequence with at least 95% similarity to the sequence of SEQ ID NO: 4.

The present invention further relates to fragments, analogs and derivatives of a polypeptide of the invention, where the “fragment,” “derivative” and “analog” retains essentially the same biological function or activity as a polypeptide as set forth in any one of SEQ ID NOS: 2 to 4. Thus, the fragment, analog or derivative can have a melting temperature of at least 40° C., an EC50 equal to or below the EC50 of wild-type PDGFB (e.g. a polypeptide of SEQ ID NO: 1) when the EC50 of the fragment, derivative or analog and the EC50 of the wild-type polypeptide is determined under the same conditions, and/or is soluble.

The polypeptides and polynucleotides of the present invention are preferably provided in an isolated form, and preferably are purified to homogeneity. “Isolated” means that the polypeptide or polynucleotide is separated from its natural environment.

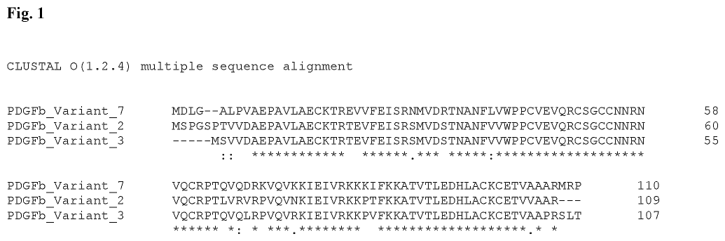

In some embodiments, the polypeptide of the invention comprises a sequence which is a consensus sequence of the alignment shown in FIG. 1.

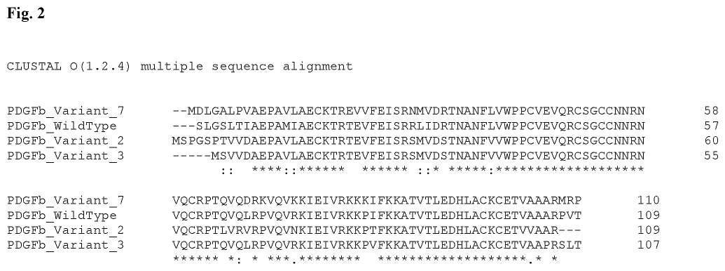

In some embodiments, the polypeptide of the invention comprises a sequence which is a consensus sequence of the alignment shown in FIG. 2.

In some embodiments, the consensus sequence comprises the amino acid residues in common between all of SEQ ID NOs: 2-4. In some embodiments, the consensus sequence comprises the amino acid residues in common between all of SEQ ID NOs: 2-4 that differ from SEQ ID NO: 1.

In some embodiments, the polypeptide of the invention comprises a sequence which is a consensus sequence of the alignment of one of FIG. 1 or FIG. 2, wherein, for residues which are not in common between all of SEQ ID NOs: 2-4, the polypeptide of the invention comprises one of the residues of one of SEQ ID NOs: 2-4 at that position.

In some embodiments of the invention, the minimum length of the polypeptide of the invention is 80% the length of the wild-type PDGFB sequence of SEQ ID NO: 1. In some embodiments of the invention, the minimum length of the polypeptide of the invention is 85% the length of the wild-type PDGFB sequence of SEQ ID NO: 1. In some embodiments of the invention, the minimum length of the polypeptide of the invention is 90% the length of the wild-type PDGFB sequence of SEQ ID NO: 1. In some embodiments of the invention, the minimum length of the polypeptide of the invention is 95% the length of the wild-type PDGFB sequence of SEQ ID NO: 1.

In some embodiments of the invention, the maximum length of the polypeptide of the invention is twice as long as the length of the wild-type PDGFB sequence of SEQ ID NO: 1. In some embodiments of the invention, the maximum length of the polypeptide of the invention is 80% more than the length of the wild-type PDGFB sequence of SEQ ID NO: 1. In some embodiments of the invention, the maximum length of the polypeptide of the invention is 70% more than the length of the wild-type PDGFB sequence of SEQ ID NO: 1. In some embodiments of the invention, the maximum length of the polypeptide of the invention is 60% more than the length of the wild-type PDGFB sequence of SEQ ID NO: 1. In some embodiments of the invention, the maximum length of the polypeptide of the invention is 50% more than the length of the wild-type PDGFB sequence of SEQ ID NO: 1. In some embodiments of the invention, the maximum length of the polypeptide of the invention is 40% more than the length of the wild-type PDGFB sequence of SEQ ID NO: 1. In some embodiments of the invention, the maximum length of the polypeptide of the invention is 30% more than the length of the wild-type PDGFB sequence of SEQ ID NO: 1. In some embodiments of the invention, the maximum length of the polypeptide of the invention is 20% more than the length of the wild-type PDGFB sequence of SEQ ID NO: 1. In some embodiments of the invention, the maximum length of the polypeptide of the invention is 10% more than the length of the wild-type PDGFB sequence of SEQ ID NO: 1.

In some embodiments, the polypeptide of the invention is the same length as the wild-type PDGFB sequence of SEQ ID NO: 1.

In some embodiments, the polypeptide of the invention has a length between 50% shorter and 50% longer than the wild-type PDGFB sequence of SEQ ID NO: 1. In some embodiments, the polypeptide of the invention has a length between 40% shorter and 40% longer than the wild-type PDGFB sequence of SEQ ID NO: 1. In some embodiments, the polypeptide of the invention has a length between 30% shorter and 30% longer than the wild-type PDGFB sequence of SEQ ID NO: 1. In some embodiments, the polypeptide of the invention has a length between 20% shorter and 20% longer than the wild-type PDGFB sequence of SEQ ID NO: 1. In some embodiments, the polypeptide of the invention has a length between 10% shorter and 10% longer than the wild-type PDGFB sequence of SEQ ID NO: 1. In some embodiments, the polypeptide of the invention has a length between 5% shorter and 5% longer than the wild-type PDGFB sequence of SEQ ID NO: 1. In some embodiments, the polypeptide of the invention has a length between 2% shorter and 2% longer than the wild-type PDGFB sequence of SEQ ID NO: 1.

### Nucleic Acid Sequences of the Invention

In some embodiments, the invention provides a nucleic acid sequence encoding any of the polypeptides disclosed herein.

In some embodiments, the invention provides a nucleic acid sequence encoding the polypeptide of any one of SEQ ID NOs: 2-4.

In some embodiments, the invention provides a nucleic acid sequence having at least 80% identity with the nucleic acid sequence of any one of SEQ ID NOs: 5-7. In some embodiments, the invention provides a nucleic acid sequence having at least 85% identity with the nucleic acid sequence of any one of SEQ ID NOs: 5-7. In some embodiments, the invention provides a nucleic acid sequence having at least 90% identity with the nucleic acid sequence of any one of SEQ ID NOs: 5-7. In some embodiments, the invention provides a nucleic acid sequence having at least 92% identity with the nucleic acid sequence of any one of SEQ ID NOs: 5-7. In some embodiments, the invention provides a nucleic acid sequence having at least 95% identity with the nucleic acid sequence of any one of SEQ ID NOs: 5-7. In some embodiments, the nucleic acid sequence of the invention encodes a PDGF polypeptide with increased stability compared to the PDGFB polypeptide of SEQ ID NO: 1.

In some embodiments, the invention provides a nucleic acid sequence having at least 96% identity with the nucleic acid sequence of any one of SEQ ID NOs: 5-7. In some embodiments, the invention provides a nucleic acid sequence having at least 97% identity with the nucleic acid sequence of any one of SEQ ID NOs: 5-7. In some embodiments, the invention provides a nucleic acid sequence having at least 98% identity with the nucleic acid sequence of any one of SEQ ID NOs: 5-7. In some embodiments, the invention provides a nucleic acid sequence having at least 99% identity with the nucleic acid sequence of any one of SEQ ID NOs: 5-7.

In a preferred embodiment, the invention provides a nucleic acid sequence having at least 92% identity with the nucleic acid sequence of any one of SEQ ID NOs: 5-7, which encodes a PDGF polypeptide with increased stability compared to the PDGFB polypeptide of SEQ ID NO: 1.

In a preferred embodiment, the invention provides a nucleic acid sequence having at least 95% identity with the nucleic acid sequence of any one of SEQ ID NOs: 5-7, which encodes a PDGF polypeptide with increased stability compared to the PDGFB polypeptide of SEQ ID NO: 1.

The nucleic acid molecule of the invention can be mRNA, DNA, cDNA or genomic DNA.

In some embodiments, the invention provides a nucleic acid sequence that hybridises with the complement of the nucleic acid sequence of any one of SEQ ID NOs: 5-7.

In some embodiments, the invention provides a nucleic acid sequence which is the reverse complement of a nucleic acid sequence of the invention.

In some embodiments, the invention provides a vector comprising a polynucleotide of the present invention.

In some embodiments, the invention provides a cell comprising the polypeptide or nucleic acid sequence described herein. The cell may be genetically engineered to express the vector of the invention.

In some embodiments, the cell is a bacteria cell, a yeast cell, a plant cell, an insect cell or a mammalian cell. In some embodiments, the bacteria cell is an Escherichia coli (E. coli) cell or a Corynebacterium glutamicum cell. In some embodiments, the cell is an E. coli cell. In some embodiments, the cell is a yeast cell.

In some embodiments, the invention provides use of the polypeptide described herein in a cell culture medium. In some embodiments, the cell culture medium is an animal cell culture medium. The cell cultured medium may be serum-free.

A cell culture medium is a medium used for the viability, growth and/or storage of cells. In some embodiments, the cell cultured medium of the invention is used for culture of fibroblasts, myoblasts, adipocytes, mesenchymal stem cells or iPSCs.

A cell culture medium of the invention can additionally comprise one or more additional growth factors, serum or serum replacement, one or more hormones, one or more antibiotics, one or more trace elements and/or one or more antioxidants.

In some embodiments, the invention provides a method of growing a cell, wherein the method comprises cultivating the cell in a culture medium containing the polypeptide described herein. In some embodiments, the cell is an animal cell. In some embodiments the cell culture medium is an animal cell culture medium.

In some embodiments, the invention provides a method of preparing a polypeptide described herein. In some embodiments, the method comprises recombinant production and synthesis of a polypeptide described herein. In some embodiments, the method is a cell-free method or a method which utilises cells.

In some embodiments, the method comprises cultivating a cell described herein under conditions which allow for expression of a polypeptide described herein and, optionally, recovering the expressed polypeptide.

In some embodiments, the method comprises cell-free protein synthesis.

In some embodiments, the method comprises i) culturing a nucleic acid sequence of the invention in a cell lysate with ribosomal activity and ii) synthesising a protein encoded by the nucleic acid with a cell-free protein synthesis reaction.

Cell-free protein synthesis is described, for example, in Gregorio et al, “A User's Guide to Cell-Free Protein Synthesis”, Methods Protoc., 2019, the contents of which are incorporated by reference.

It will be understood that particular embodiments described herein are shown by way of illustration and not as limitations of the invention. The principal features of this invention can be employed in various embodiments without departing from the scope of the invention. Those skilled in the art will recognise, or be able to ascertain using no more than routine study, numerous equivalents to the specific procedures described herein. Such equivalents are considered to be within the scope of this invention and are covered by the claims. All publications mentioned in the specification are indicative of the level of skill of those skilled in the art to which this invention pertains.

All publications are herein incorporated by reference to the same extent as if each individual publication was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the inherent variation of error for the feature in the below.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps

The term “or combinations thereof” as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

Any part of this disclosure may be read in combination with any other part of the disclosure, unless otherwise apparent from the context.

All of the polypeptides, nucleic acids and media disclosed and claimed herein can be made and executed without undue experimentation in light of the present disclosure. While the invention has been described in terms of preferred embodiments, it will be apparent to those of skill in the art that variations may be applied to the polypeptides, nucleic acids, media and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

The present invention is described in more detail in the following non limiting exemplification.

### EXAMPLES

The following examples will be useful in demonstrating the present invention.

### Example 1: Polypeptide Variants and Alignments

The following 3 variants are disclosed herein:

Variant 2 (SEQ ID NO: 2):
MSPGSPTVVDAEPAVLAECKTRTEVFEISRSMVDSTNANFVVWPP
CVEVQRCSGCCNNRNVQCRPTLVRVRPVQVNKIEIVRKKPTFKKA
TVTLEDHLACKCETVVAAR
Variant 3 (SEQ ID NO: 3):
MSVVDAEPAVLAECKTRTEVFEISRSMVDSTNANFVVWPPCVEVQ
RCSGCCNNRNVQCRPTQVQLRPVQVRKIEIVRKKPVFKKATVTLE
DHLACKCETVAAPRSLT
Variant 7 (SEQ ID NO: 4):
MDLGALPVAEPAVLAECKTREVVFEISRNMVDRTNANFLVWPPCV
EVQRCSGCCNNRNVQCRPTQVQDRKVQVKKIEIVRKKKIFKKATV
TLEDHLACKCETVAAARMRP

The nucleic acid sequences equivalent to SEQ ID NOs: 2-4 (variants 2, 3 and 7) are provided as SEQ ID NOs: 5-7 and shown below:

Variant 2 (SEQ ID NO: 5):
ATGTCTCCCGGTTCACCGACAGTTGTGGATGCAGAACCAGCTGTG
CTGGCTGAGTGTAAAACCCGAACGGAAGTTTTCGAAATCAGCCGC
TCTATGGTAGATTCGACTAATGCCAATTTTGTGGTGTGGCCCCCT
TGTGTTGAAGTTCAGCGTTGCTCCGGTTGTTGTAACAACAGAAAC
GTTCAATGTCGGCCAACGCTGGTGCGTGTTAGACCTGTTCAGGTA
AATAAGATAGAAATTGTCCGCAAGAAGCCGACGTTTAAAAAAGCG
ACCGTGACGCTGGAAGATCACCTTGCCTGTAAATGTGAAACAGTG
GTGGCAGCGCGG
Variant 3 (SEQ ID NO: 6):
ATGAGCGTAGTTGACGCAGAACCAGCAGTGTTGGCCGAATGTAAA
ACCCGCACAGAAGTGTTCGAAATTAGCCGTAGCATGGTGGATAGT
ACAAATGCAAATTTTGTCGTTTGGCCGCCGTGCGTTGAGGTACAA
CGCTGTAGTGGTTGCTGCAATAATAGAAACGTTCAGTGCCGTCCC
ACCCAGGTCCAGTTGCGCCCCGTCCAAGTTCGTAAGATTGAAATT
GTACGCAAAAAACCGGTGTTCAAGAAAGCGACAGTTACGCTCGAA
GATCACTTGGCCTGCAAATGTGAAACGGTTGCAGCGCCGAGAAGC
TTGACC
Variant 7 (SEQ ID NO: 7):
ATGGATTTAGGAGCACTCCCTGTTGCTGAACCGGCCGTCCTTGCA
GAGTGTAAAACTCGGGAAGTCGTTTTCGAGATTTCCCGCAATATG
GTGGATCGTACTAATGCAAATTTTCTGGTGTGGCCGCCTTGCGTT
GAGGTGCAGCGGTGTTCAGGTTGTTGTAATAATCGCAATGTGCAG
TGCCGACCTACTCAGGTCCAGGATAGAAAAGTTCAGGTCAAAAAA
ATAGAGATTGTCCGGAAAAAGAAGATATTTAAGAAAGCGACTGTG
ACATTAGAAGACCATTTGGCCTGCAAATGTGAAACAGTGGCGGCG
GCCCGGATGCGCCCT

Alignments were generated using the Clustal Omega Multiple Sequence Alignment tool from EMBL-EBI. Version O (1.2.4) was used, retrieved from “https://www.ebi.ac.uk/Tools/msa/clustalo/”.

Alignment of the amino acid sequence of variants 2, 3 and 7 (SEQ ID NOs: 2-4) is shown in FIG. 1.

Alignment of the amino acid sequence of variants 2, 3 and 7 (SEQ ID NOs: 2-4) and the wild-type PDGFB sequence (SEQ ID NO: 1) is shown in FIG. 2.

A number of differences can be seen that all 3 variants have in common, but that vary from the wild-type PDGFB, such as the 130V substitution.

### Example 2: Polypeptide Expression

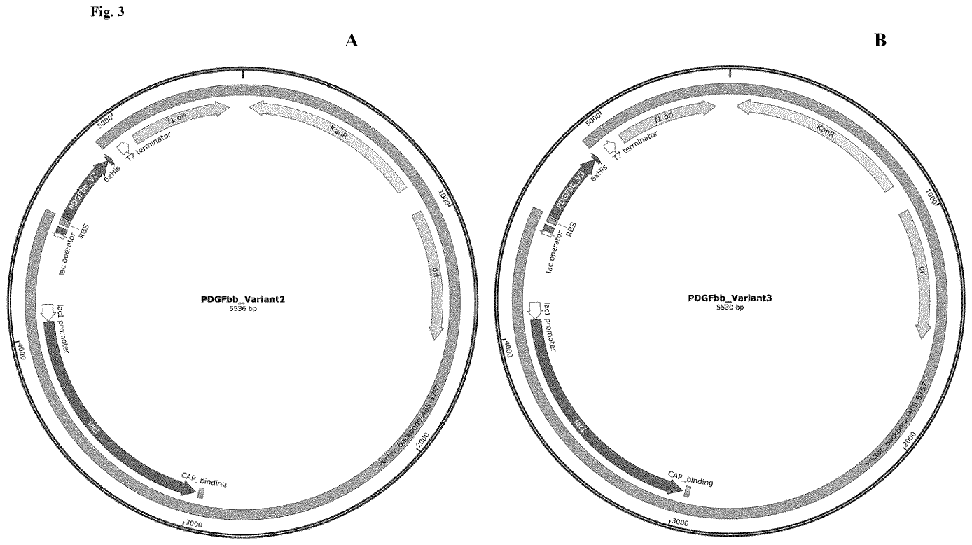

Plasmid inserts for each of the 3 variants of SEQ ID NOs: 2-4 were generated, by converting primary amino acid sequences to DNA sequences via codon optimisation. These plasmids are shown in FIGS. 3A-3C.

The insert containing the engineered variants were digested with BsaI and ligated into a modified pET-28a (+) backbone, pGT94. E. coli strain BL21 (DE3) was used to transform 5 ul of the reaction mix. Electroporation was carried out using the protocols provided for the MicroPulser Electroporator (Bio-Rad). Single colonies were picked, and the sequence was verified via Sanger sequencing (Azenta).

Overnight culture was prepared using 10 ml of LB supplemented with 50 ug/ml of Kanamycin. The starter culture was diluted 1:100 into 50 ml of autoinducible expression media (Auto Induction Media, Formedium) for 24 hours, 250 rpm at 30° C. The culture was pelleted at 10,000×g for 10 min at 4° C. and stored at −20° C.

Lysis was carried out on in buffer A (50 mM Tris, 300 mM NaCl, pH 8) supplemented with 5% Glycerol and 1 mM DTT, and protease inhibitor cocktail (Roche). Sonication (Branson Ultrasonics) was performed at 45%, at 10 seconds on/off. The crude lysate was pelleted at 10,000×g for 30 minutes at 4° C.

The insoluble fraction of the crude lysate was resolubilized in buffer D1 (100 mM NaH₂PO₄, 10 mM Tris, 8M Urea, 10 mM Imidazole, 1 mM DTT, 0.05% Tween-20, pH 8) at 4° C. The crude lysate was clarified at 1000×g for 5 minutes at 4° C. Ni-charged magnetic beads (Genescript) were pre-equilibrated with Buffer D1 and were incubated with the clarified lysate at 4° C. prior to affinity chromatography. Buffer D2 (100 mM NaH₂PO₄, 150 mM NaCl, 8M Urea, 20 mM Imidazole, pH 8) was used to wash the beads for 7 column volumes, two times, followed by Buffer D3 (50 mM NaH₂PO₄, 500 mM NaCl, 20 mM Imidazole, pH 8) for 10 column volumes, five times. The protein was eluted by adding and incubating for 5 minutes in 2 column volumes of Buffer E1 (50 mM NaHPO₄, 500 mM NaCl, 250 mM Imidazole, pH 8) and a subsequent elution at 1.5 column volume in E1. 10 ul of eluant was analysed with SDS-PAGE (40 mA, 60 min, Bis-Tris/MES). The process was carried out on ice.

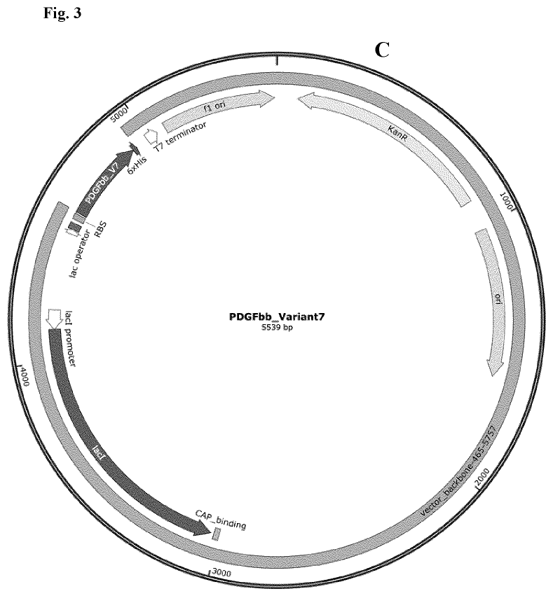

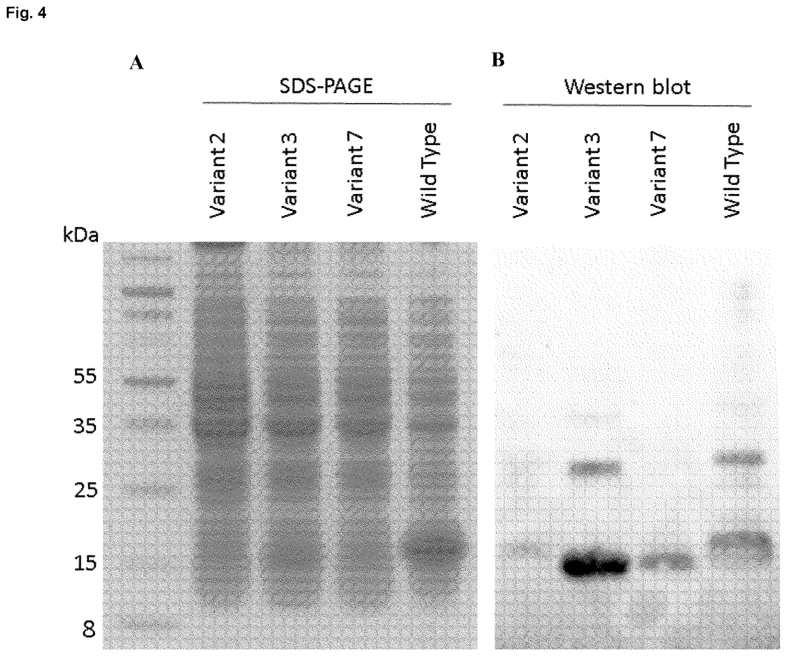

The results of the SDS-PAGE are shown in FIG. 4A.

Approximately 20 ug of protein was loaded on Bis-Tris gel (Genscript), then blotted (100V, 1 hour, 4° C.) on to a nitrocellulose membrane using the Trans-Blot module (Bio-rad). The membrane was blocked with TBS supplemented with 5% skimmed milk powder. Variants were detected using a 1:1000 dilution of the Mouse anti-His primary antibody (Biolegend) followed by 1:2500 dilution of the horseradish peroxidase (HRP) conjugated Goat anti-mouse secondary antibody (Biolegend). Bands were identified using the ECL Western Blotting Substrate (Promega) as per manufacturer instructions.

The results of the western blot are shown in FIG. 4B.

As can be seen from FIGS. 4A and 4B, all variants are successfully expressed.

### Example 3: Polypeptide Bioactivity

Bioactivity was assessed using ONE-Glo Luciferase assay (Promega). HEK293T cells were seeded at 5000 cells/well in a clear, flat bottom 96-well plate and transfected with pGL4.33 (Promega) containing a Serum Response element (SRE) at 2:1 ratio with Viafect transfection reagent (Promega) as per manufacturer's instructions. Cells were starved overnight in DMEM-F12 supplemented with 0.5% FBS and 1% Penicillin Streptomycin. Elutions of purified protein variants were serially diluted in DMEM-F12 supplemented with 1% Penicillin streptomycin and treated for 6 hours with DMEM-F12 supplemented with 20% FBS+20 ng/ml PMA as positive control and DMEM-F12 as negative control. One-Glo buffer and substrate was reconstituted and added to the samples as per manufacturer's instructions. All experiments were performed in triplicate.

For accurate quantitation, 10 μl of the diluted samples were diluted in 90 ul DMEM F12 and analysed using the His-Tag ELISA Detection kit (Genscript) as per manufacturer's instructions. Luminescence of the samples were measured. Relative luminescence intensities (RLU) were normalized using the negative control. Dose response curves were plotted and EC50 values were calculated using the Graphpad-Prism software.

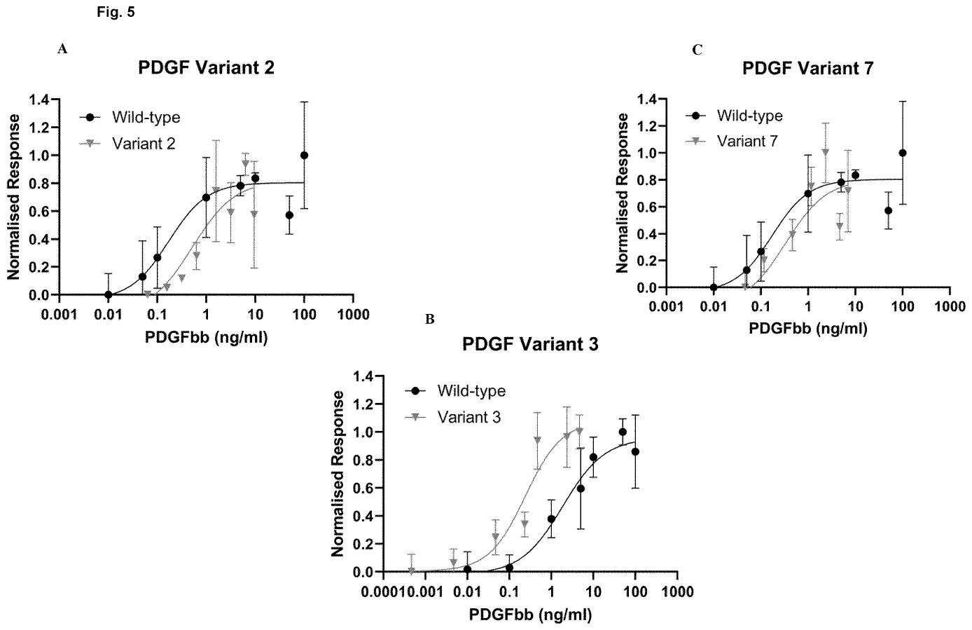

The results are shown in FIGS. 5A-5C.

As can be seen from the graphs, all the variants presented show bioactivity. The bioactivity is comparable to that of wild-type PDGFB. Variant 3 shows higher affinity for the receptor (i.e., enhanced bioactivity) compared to wild-type PDGFB.

### Example 4: Polypeptide Solubility-SDS-PAGE and Western Blot

To assess solubility, 100 ul of the crude lysate and clarified lysate was collected before and after pelleting, during the lysis step. Total protein content was quantified using Bradford assay (Bio-rad) using Bovine serum albumin as a standard. 30 ug protein was loaded onto each well and analysed using western blot. The ratio of soluble protein to total protein was calculated by quantifying the intensity of the band in both fraction using ImageJ.

The ratio was calculated using the formula:

$$Soluble fraction ( \% ) = \frac{{Intensity}_{clarified lysate}}{{Intensity}_{crude lysate}} \times 100$$

The results are shown in Table 1.

TABLE 1
Results of solubility analysis for the PDGF polypeptides of SEQ
ID NOs: 2-4 and the wild-type PDGFB sequence (SEQ ID NO: 1).

| | Intensity (RLU) | | Fraction (%) | |
|---|---|---|---|---|
| Variant | Crude Lysate | Clarified lysate | Soluble | Insoluble |
| Variant 2 | 16145.3 | 6487.08 | 40.18 | 59.82 |
| SEQ ID NO: 2 | | | | |
| Variant 3 | 58463.19 | 31432.49 | 53.76 | 46.24 |
| SEQ ID NO: 3 | | | | |
| Variant 7 | 36815.12 | 4974.03 | 13.51 | 86.49 |
| SEQ ID NO: 4 | | | | |
| WT | 27175.27 | 0 | 0 | 100 |
| SEQ ID NO: 1 | | | | |

Measurements of zero in Table 1 indicate levels beyond the detection limit.

Table 1 shows all variants to be soluble. All variants show improved solubility compared to wild-type PDGFB, with variant 3 having the greatest solubility.

### Example 5: Polypeptide thermostability

Thermostability of the polypeptides was predicted using property predictors trained on public datasets of PDGFB variants.

The results are shown in Table 2.

TABLE 2
Results of in silico thermostability predictions
for the PDGF polypeptides of SEQ ID NOs: 2-4 and
the wild-type PDGFB sequence (SEQ ID NO: 1).

| | Thermostability |
|---|---|
| | (melting |
| Variant | temperature) (° C.) |
| Variant | Predicted |
| Variant 2 | 50.90 |
| SEQ ID NO: 2 | |
| Variant 3 | 51.95 |
| SEQ ID NO: 3 | |
| Variant 7 | 51.95 |
| SEQ ID NO: 4 | |
| WT | 49.99 |
| SEQ ID NO: 1 | |

All 3 variants also show increased thermostability compared to wild-type PDGFB. The highest increase in thermostability is seen in variants 3 and 7.

These data, taken in conjunction with the solubility and bioactivity data, show that the polypeptides with the sequences of SEQ ID NOs: 2-4 have improved properties.

### Example 6: Stability-Determined by Measuring Bioactivity Over Time

Bioactivity of the polypeptide variants can be assessed over time to determine stability, including thermostability. This assay confirms that the variants retain bioactivity at appropriate levels. Purified protein variants were added to DMEM-F12 supplemented with 1% Penicillin and Streptomycin. Protein variants were incubated at 37° C. for ten days and samples were collected every 24 hours. Bioactivity of each sample was measured using the ONE-Glo Luciferase assay (Promega), previously described. Bioactivity of each protein sample was compared by producing an EC50 derived from a sigmoidal curve to the wild-type protein incubated under identical conditions.

SEQUENCES

| SEQ | | | |
|---|---|---|---|
| ID | | | |
| NO: | Description | Sequence | Length |
| 1 | Human wild- | SLGSLTIAEPAMIAECKTRTEVFEISRRLIDRTNANFLVW | 109 |
| | type PDGFB | PPCVEVQRCSGCCNNRNVQCRPTQVQLRPVQVRKIEIVRK | |
| | (aa) | KPIFKKATVTLEDHLACKCETVAAARPVT | |
| 2 | Variant 2 (aa) | MSPGSPTVVDAEPAVLAECKTRTEVFEISRSMVDSTNAN | 109 |
| | | FVVWPPCVEVQRCSGCCNNRNVQCRPTLVRVRPVQVNKI | |
| | | EIVRKKPTFKKATVTLEDHLACKCETVVAAR | |
| 3 | Variant 3 (aa) | MSVVDAEPAVLAECKTRTEVFEISRSMVDSTNANFVVWP | 107 |
| | | PCVEVQRCSGCCNNRNVQCRPTQVQLRPVQVRKIEIVRK | |
| | | KPVFKKATVTLEDHLACKCETVAAPRSLT | |
| 4 | Variant 7 (aa) | MDLGALPVAEPAVLAECKTREVVFEISRNMVDRTNANFL | 110 |
| | | VWPPCVEVQRCSGCCNNRNVQCRPTQVQDRKVQVKKIEI | |
| | | VRKKKIFKKATVTLEDHLACKCETVAAARMRP | |
| 5 | Variant 2 (nt) | ATGTCTCCCGGTTCACCGACAGTTGTGGATGCAGAACC | 327 |
| | | AGCTGTGCTGGCTGAGTGTAAAACCCGAACGGAAGTTT | |
| | | TCGAAATCAGCCGCTCTATGGTAGATTCGACTAATGCC | |
| | | AATTTTGTGGTGTGGCCCCCTTGTGTTGAAGTTCAGCG | |
| | | TTGCTCCGGTTGTTGTAACAACAGAAACGTTCAATGTC | |
| | | GGCCAACGCTGGTGCGTGTTAGACCTGTTCAGGTAAAT | |
| | | AAGATAGAAATTGTCCGCAAGAAGCCGACGTTTAAAAA | |
| | | AGCGACCGTGACGCTGGAAGATCACCTTGCCTGTAAAT | |
| | | GTGAAACAGTGGTGGCAGCGCGG | |
| 6 | Variant 3 (nt) | ATGAGCGTAGTTGACGCAGAACCAGCAGTGTTGGCCG | 321 |
| | | AATGTAAAACCCGCACAGAAGTGTTCGAAATTAGCCGT | |
| | | AGCATGGTGGATAGTACAAATGCAAATTTTGTCGTTTG | |
| | | GCCGCCGTGCGTTGAGGTACAACGCTGTAGTGGTTGCT | |
| | | GCAATAATAGAAACGTTCAGTGCCGTCCCACCCAGGTC | |
| | | CAGTTGCGCCCCGTCCAAGTTCGTAAGATTGAAATTGT | |
| | | ACGCAAAAAACCGGTGTTCAAGAAAGCGACAGTTACGC | |
| | | TCGAAGATCACTTGGCCTGCAAATGTGAAACGGTTGCA | |
| | | GCGCCGAGAAGCTTGACC | |
| 7 | Variant 7 (nt) | ATGGATTTAGGAGCACTCCCTGTTGCTGAACCGGCCGT | 330 |
| | | CCTTGCAGAGTGTAAAACTCGGGAAGTCGTTTTCGAGA | |
| | | TTTCCCGCAATATGGTGGATCGTACTAATGCAAATTTT | |
| | | CTGGTGTGGCCGCCTTGCGTTGAGGTGCAGCGGTGTTC | |
| | | AGGTTGTTGTAATAATCGCAATGTGCAGTGCCGACCTA | |
| | | CTCAGGTCCAGGATAGAAAAGTTCAGGTCAAAAAAATA | |
| | | GAGATTGTCCGGAAAAAGAAGATATTTAAGAAAGCGAC | |
| | | TGTGACATTAGAAGACCATTTGGCCTGCAAATGTGAAA | |
| | | CAGTGGCGGCGGCCCGGATGCGCCCT | |

1. A platelet-derived growth factor (PDGF) polypeptide comprising at least one modification selected from the group consisting of an amino acid substitution, an amino acid deletion, an amino acid insertion, and a combinations thereof relative to the sequence set forth in SEQ ID NO: 1, wherein the modified PDGF polypeptide exhibits increased stability, solubility and/or bioactivity compared to the PDGFB polypeptide of SEQ ID NO: 1.

2. The PDGF polypeptide of claim 1, wherein the polypeptide comprises a sequence of any one of SEQ ID NOs: 2-4, or a sequence with at least 80%, 85%, 90%, 92% or 95% sequence similarity to any one of SEQ ID NOs: 2-4, wherein the polypeptide has a melting temperature of at least 40° C.

3. The PDGF polypeptide of claim 1, wherein the polypeptide comprises a sequence of SEQ ID NO: 2 or SEQ ID NO: 4 or a sequence with at least 80%, 85%, 90%, 92% or 95% sequence similarity to SEQ ID NO: 2 or SEQ ID NO: 4, wherein the polypeptide has a melting temperature of at least 40° C.

4. The PDGF polypeptide of claim 1, wherein the polypeptide comprises a sequence of SEQ ID NO: 2 or a sequence with at least 80%, 85%, 90%, 92% or 95% sequence similarity to SEQ ID NO: 2, wherein the polypeptide has a melting temperature of at least 40° C.

5. The PDGF polypeptide of claim 1, wherein the polypeptide comprises a sequence of SEQ ID NO: 3 or a sequence with at least 80%, 85%, 90%, 92% or 95% sequence similarity to SEQ ID NO: 3, wherein the polypeptide has a melting temperature of at least 40° C.

6. The PDGF polypeptide of claim 1, wherein the polypeptide comprises a sequence of SEQ ID NO: 4 or a sequence with at least 80%, 85%, 90%, 92% or 95% sequence similarity to SEQ ID NO: 4, wherein the polypeptide has a melting temperature of at least 40° C.

7. The PDGF polypeptide of claim 1, wherein the polypeptide comprises a sequence of SEQ ID NO: 2 or a sequence with at least 80%, 85%, 90%, 92% or 95% sequence similarity to SEQ ID NO: 2, wherein the polypeptide is more bioactive than the PDGFB polypeptide of SEQ ID NO: 1.

8. The PDGF polypeptide of claim 1, wherein the polypeptide comprises a sequence of SEQ ID NO: 3 or a sequence with at least 80%, 85%, 90%, 92% or 95% sequence similarity to SEQ ID NO: 3, wherein the polypeptide is more bioactive than the PDGFB polypeptide of SEQ ID NO: 1.

9. The PDGF polypeptide of claim 1, wherein the polypeptide comprises a sequence of SEQ ID NO: 4 or a sequence with at least 80%, 85%, 90%, 92% or 95% sequence similarity to SEQ ID NO: 4, wherein the polypeptide is more bioactive than the PDGFB polypeptide of SEQ ID NO: 1.

10. The PDGF polypeptide of claim 1, wherein the polypeptide is soluble and capable of binding a platelet-derived growth factor receptor (PDGFR), optionally PDGFRB.

11. A nucleic acid comprising a sequence selected from the group consisting of:
(a) a nucleic acid sequence encoding a PDGF polypeptide of claim 1;
(b) a nucleic acid sequence having at least 80% similarity with the nucleic acid sequence of any one of SEQ ID NOs: 5-7 and encoding a PDGF polypeptide of claim 1;
(c) a nucleic acid sequence hybridizing with the complement of the nucleic acid sequence of any one of SEQ ID NOs: 5-7 and encoding a PDGF polypeptide of claim 1; and
(d) a nucleic acid sequence which is the reverse complement of a nucleic acid sequence as defined in (a), (b) or (c).

12. A cell comprising the PDGF polypeptide or nucleic acid sequence of claim 1.

13. The cell of claim 12, wherein the cell is a bacteria cell, a yeast cell, a plant cell, an insect cell, or a mammalian cell.

14. A method of preparing a PDGF polypeptide, wherein the method comprises cultivating the cell of claim 12 under conditions which allow for expression of said polypeptide and, optionally, recovering the expressed polypeptide.

15. A method of preparing a PDGF polypeptide, wherein the method comprises i) culturing a nucleic acid sequence of claim 11 in a cell lysate with ribosomal activity, and ii) synthesising a protein encoded by the nucleic acid with a cell-free protein synthesis reaction.

16. A method of growing an animal cell, wherein the method comprises cultivating the animal cell in a culture medium containing the PDGF polypeptide of claim 1.

17. (canceled)


SEQUENCE LISTING (7 sequences)

SEQ ID NO: 1 - protein, 109 aa, Homo sapiens
SLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVT

SEQ ID NO: 2 - protein, 109 aa, synthetic construct
MSPGSPTVVDAEPAVLAECKTRTEVFEISRSMVDSTNANFVVWPPCVEVQRCSGCCNNRNVQCRPTLVRVRPVQVNKIEIVRKKPTFKKATVTLEDHLACKCETVVAAR

SEQ ID NO: 3 - protein, 107 aa, synthetic construct
MSVVDAEPAVLAECKTRTEVFEISRSMVDSTNANFVVWPPCVEVQRCSGCCNNRNVQCRPTQVQLRPVQVRKIEIVRKKPVFKKATVTLEDHLACKCETVAAPRSLT

SEQ ID NO: 4 - protein, 110 aa, synthetic construct
MDLGALPVAEPAVLAECKTREVVFEISRNMVDRTNANFLVWPPCVEVQRCSGCCNNRNVQCRPTQVQDRKVQVKKIEIVRKKKIFKKATVTLEDHLACKCETVAAARMRP

SEQ ID NO: 5 - DNA, 327 bp, synthetic construct
  source 1..327: mol_type = other DNA
atgtctcccggttcaccgacagttgtggatgcagaaccagctgtgctggctgagtgtaaaacccgaacggaagttttcgaaatcagccgctctatggtagattcgactaatgccaattttgtggtgtggcccccttgtgttgaagttcagcgttgctccggttgttgtaacaacagaaacgttcaatgtcggccaacgctggtgcgtgttagacctgttcaggtaaataagatagaaattgtccgcaagaagccgacgtttaaaaaagcgaccgtgacgctggaagatcaccttgcctgtaaatgtgaaacagtggtggcagcgcgg

SEQ ID NO: 6 - DNA, 321 bp, synthetic construct
  source 1..321: mol_type = other DNA
atgagcgtagttgacgcagaaccagcagtgttggccgaatgtaaaacccgcacagaagtgttcgaaattagccgtagcatggtggatagtacaaatgcaaattttgtcgtttggccgccgtgcgttgaggtacaacgctgtagtggttgctgcaataatagaaacgttcagtgccgtcccacccaggtccagttgcgccccgtccaagttcgtaagattgaaattgtacgcaaaaaaccggtgttcaagaaagcgacagttacgctcgaagatcacttggcctgcaaatgtgaaacggttgcagcgccgagaagcttgacc

SEQ ID NO: 7 - DNA, 330 bp, synthetic construct
  source 1..330: mol_type = other DNA
atggatttaggagcactccctgttgctgaaccggccgtccttgcagagtgtaaaactcgggaagtcgttttcgagatttcccgcaatatggtggatcgtactaatgcaaattttctggtgtggccgccttgcgttgaggtgcagcggtgttcaggttgttgtaataatcgcaatgtgcagtgccgacctactcaggtccaggatagaaaagttcaggtcaaaaaaatagagattgtccggaaaaagaagatatttaagaaagcgactgtgacattagaagaccatttggcctgcaaatgtgaaacagtggcggcggcccggatgcgccct